(19) United States
(12) Patent Application Publication
(10) Pub. No.: US 2019/0021866 A1
(43) Pub. Date: Jan. 24, 2019

(54) PATIENT-SPECIFIC GLENOID IMPLANT

(71) Applicant: Biomet Manufacturing, LLC, Warsaw, IN (US)

(72) Inventors: Thomas M. Vanasse, Gainesville, FL (US); Clinton E. Kehres, Warsaw, IN (US); Robert Taylor, Granger, IN (US)

(21) Appl. No.: 16/140,082

(22) Filed: Sep. 24, 2018

Related U.S. Application Data
(63) Continuation of application No. 15/053,229, filed on Feb. 25, 2016.
Application No. 15/053,229 is a continuation of application No. 14/095,565, filed on Dec. 3, 2013, now abandoned.

(51) Int. Cl. A61F 2/30; A61F 2/40; G06F 19/00; A61F 2/46; G16H 50/50; G06T 17/00

(52) U.S. Cl. CPC A61F 2/30942; A61F 2/4081; G06T 17/00; A61F 2/30767; A61F 2002/30828; A61F 2002/30322; A61F 2002/30897; A61F 2002/30822; A61F 2002/30574; A61F 2002/30326; A61F 2002/30324; A61F 2002/30892; G06F 19/00; A61F 2002/30971; A61F 2/4612; A61F 2002/30891; G16H 50/50

(57) ABSTRACT

The present disclosure describes a glenoid implant including a body and a fixation member. The body has an articular surface and a scapula-engaging surface opposite from the articular surface. At least a portion of the scapula-engaging surface is configured to mirror and conform to a surface of a scapula of a specific patient based on a three-dimensional (3D) model of the scapula. The fixation member extends from the scapula-engaging surface for fixing the glenoid implant to the scapula.

### FIELD

The present disclosure relates to implants, and more particularly, to patient-specific implants for an anatomical feature such as a glenoid.

### BACKGROUND

This section provides background information related to the present disclosure which is not necessarily prior art.

Shoulder joint reconstruction may require fixing a glenoid implant to a scapula to reproduce or replicate a glenoid cavity on the scapula. The glenoid implant may be fixed to the scapula using mounting hardware such as bone screws. Alternatively, the glenoid implant may include pegs, and holes may be formed in the scapula for receiving the pegs. The holes may be sized to yield a press or interference fit between the glenoid implant and the scapula. Shoulder joint reconstruction may also require repairing a defect in a shoulder joint such as a void in a glenoid cavity resulting from severe wear.

Current methods for reconstructing a shoulder joint may not be sufficiently accurate to reproduce the natural anatomy of the shoulder joint such as glenoid version. Typically, surgical planning for a shoulder joint reconstruction is based on two-dimensional (2D) x-rays. During the procedure, a surgeon may visually examine a defect in a glenoid cavity and attempt to fill the defect using bone graft that the surgeon shapes by hand. The surgeon may then position the glenoid implant over the bone graft and fix the glenoid implant to the scapula.

Performing shoulder joint reconstructions in the manner described above can be tedious and time consuming. In addition, a glenoid implant may not accurately replicate a glenoid cavity in its original state (e.g., before the shape of the glenoid cavity is altered due to wear). Further, it is difficult to form bone graft by hand to accurately conform to and fill a defect. Thus, the natural movement of the shoulder joint may not be reproduced.

### SUMMARY

This section provides a general summary of the disclosure, and is not a comprehensive disclosure of its full scope or all of its features.

The present disclosure describes a glenoid implant including a body and a fixation member. The body has an articular surface and a scapula-engaging surface opposite from the articular surface. At least a portion of the scapula-engaging surface is configured to mirror and conform to a surface of a scapula of a specific patient based on a three-dimensional (3D) model of the scapula. The fixation member extends from the scapula-engaging surface for fixing the glenoid implant to the scapula. Methods associated with a glenoid implant are also described.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a first perspective view of a first glenoid implant according to the principles of the present disclosure;

FIG. 2 is a second perspective view of the first glenoid implant;

FIG. 3 is a side view of the first glenoid implant;

FIG. 4 is a top view of the first glenoid implant;

FIG. 5 is a front view of the first glenoid implant;

FIG. 6 is a first perspective view of a second glenoid implant according to the principles of the present disclosure;

FIG. 7 is a second perspective view of the second glenoid implant;

FIG. 8 is a side view of the second glenoid implant;

FIG. 9 is a top view of the second glenoid implant;

FIG. 10 is a front view of the second glenoid implant;

FIG. 11 is a superior view of the second glenoid implant fixed to a scapula; and

FIG. 12 is a medial view of the second glenoid implant fixed to the scapula.

Corresponding reference numerals indicate corresponding parts throughout the several views of the drawings.

### DETAILED DESCRIPTION

Example embodiments will now be described more fully with reference to the accompanying drawings.

The present disclose describes patient-specific glenoid implants for use in shoulder joint replacement. The glenoid implants can replace or replicate an entire glenoid cavity or a portion thereof. The glenoid implants can also fill a defect in the glenoid cavity such as a void due to severe wear. The glenoid implants may include pegs, and holes may be formed in a scapula for receiving the pegs. The holes may be sized to yield a press fit between the glenoid implants and the scapula. The glenoid implants may be used for both anatomic and reverse shoulder joint replacements.

The glenoid implants can be designed and formed based on three-dimensional (3D) computer models of the anatomy of a specific patient. The 3D models may be generated based on imaging data obtained from an x-ray, magnetic resonance imaging (MRI), computed tomography (CT scan), ultrasound, or other medical scan. In the medical scan, an anatomical feature (e.g., a scapula with or without surrounding soft tissue) can be imaged to detect certain features of the anatomy (e.g., dimensions, curvature of surfaces, etc.). Software programs may be used to generate the 3D models of the patient's anatomy based on the imaging data. 3D models of patient-specific implants can be created based on the 3D models of the patient's anatomy, and the patient-specific implants can be formed based on the patient-specific implant 3D models.

The geometry, shape and orientation of the various features of the patient-specific implants can be determined during the pre-operative planning stage of the procedure in connection with the computer-assisted modeling of the patient's anatomy. During the pre-operative planning stage, custom, semi-custom or non-custom implants can be selected, and the patient-specific components can be manufactured for a specific patient with input from a surgeon or other professional associated with the surgical procedure.

Patient-specific instruments, such as drills, reamers, and/or guides, can be used to prepare anatomical features such as bone before fixing the patient-specific implants to the bone. The patient-specific implants and/or instruments can have a three-dimensional engagement surface that is complementary and made to conformingly contact the anatomical surface. Thus, the patient-specific implants and/or instruments can be configured to seat against the anatomical surface in only one position.

In the following discussion, the terms “patient-specific”, “custom-made” or “customized” are defined to apply to components, including tools, implants, portions or combinations thereof, which include certain geometric features, including surfaces, curves, or other lines, and which are made to closely conform to as mirror-images or negatives or complementary surfaces of corresponding geometric features or anatomic landmarks of a patient's anatomy obtained or gathered during a pre-operative planning stage based on 3D computer images of the corresponding anatomy reconstructed from image scans of the patient by computer imaging methods.

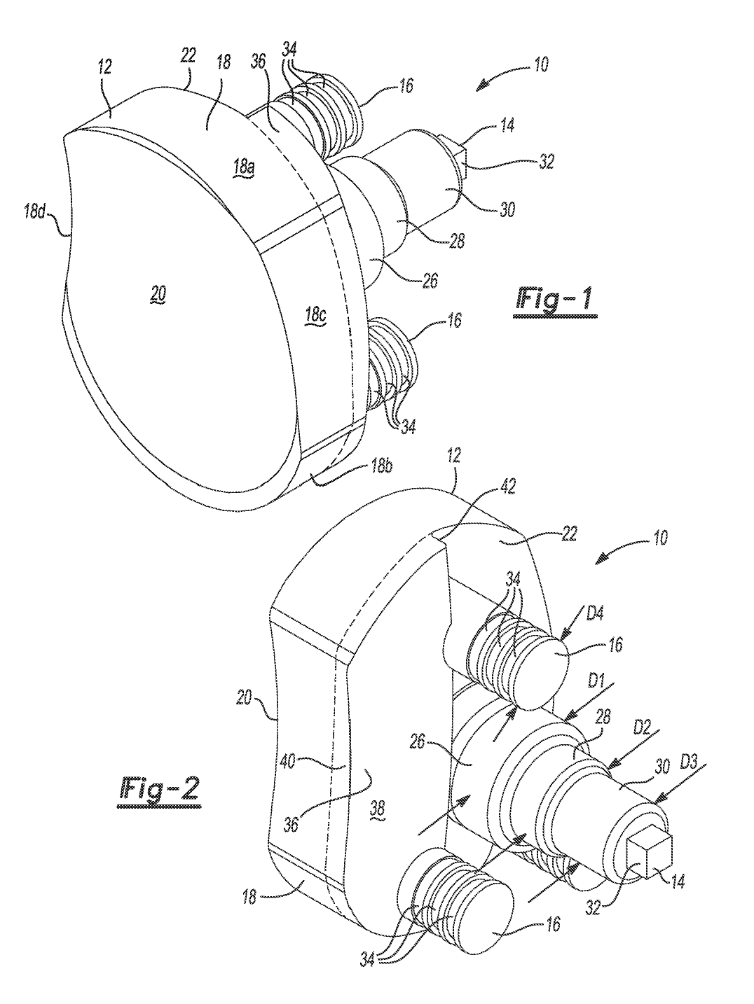

Referring now to FIGS. 1 through 5, a glenoid implant 10 is configured (e.g., sized and shaped) to replicate or replace an entire glenoid cavity or a portion thereof. The implant 10 includes a generally rectangular body 12 having a pear-shaped outline, a cylindrical central peg 14, and cylindrical peripheral pegs 16. The body 12 has a peripheral surface 18, an articular surface 20, and a scapula-engaging surface 22 opposite from the articular surface 20. The peripheral surface 18 includes superior and infererior portions 18a, 18b that are rounded (e.g., concave), and anterior and posterior portions 18c, 18d that are flat or slightly rounded (e.g., convex). The peripheral surface 18 can be patient-specific and can match or replicate a peripheral surface of a glenoid cavity of a specific patient. The central peg 14 and the peripheral pegs 16 extend from the scapula-engaging surface 22 of the body 12. Although the implant 10 is shown with three peripheral pegs, the implant 10 can include additional or fewer peripheral pegs.

The articular surface 20 is configured to partially receive and nestingly engage or articulate with the humeral head. For example, the articular surface 20 can be patient-specific and can have a concave hemispherical shape that closely conforms as mirror-image or negative or a complementary surface of the humeral head. The humeral head can be part of a natural humerus of a specific patient, or the humeral head can be part of a humeral implant. A 3D model of the humeral head can be obtained using an x-ray, MRI, CT, ultrasound or other medical scan, and the articular surface 20 can be designed (e.g., shaped, sized, contoured) based on the 3D model. If the humeral head is part of a humeral implant, the 3D model can be obtained from the CAD files used to design the humeral implant.

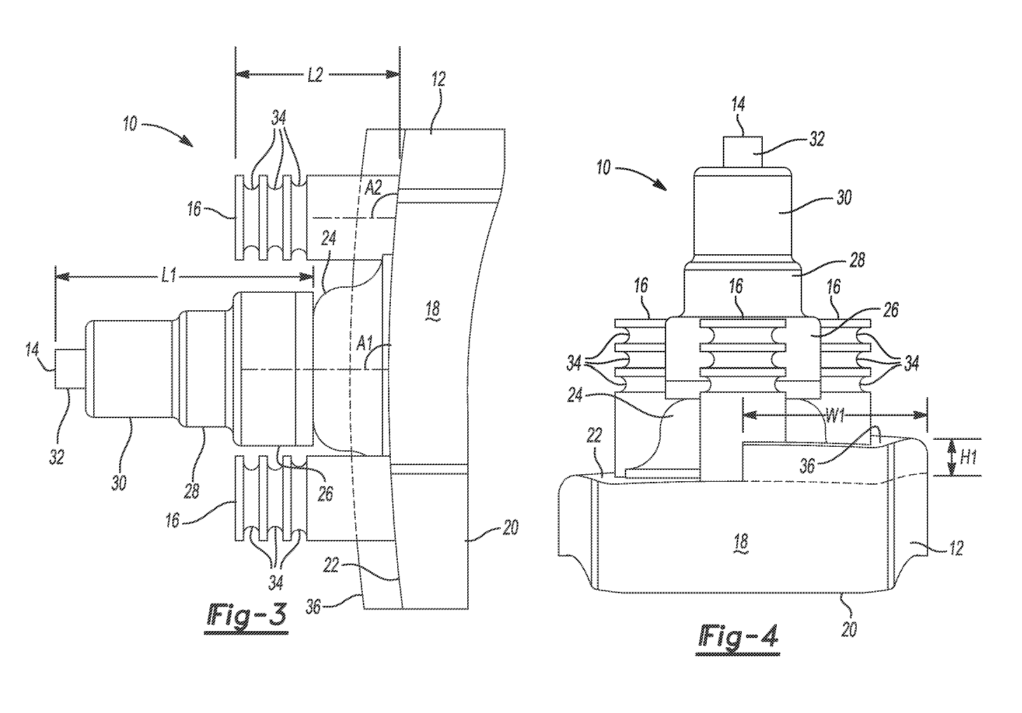

The central peg 14 and/or the peripheral pegs 16 can be formed integral with the body 12 or separate from the body 12. In one example, the peripheral pegs 16 can be formed integral with the body 12, and the central peg 14 can be formed separate from the body 12 and press fit or threaded into a blind hole in the body 12. The blind hole can be formed in a domed portion 24 (FIGS. 3 and 4) of the body 12 that extends from the scapula-engaging surface 22.

The central peg 14 includes a first portion 26, a second portion 28 that extends from the first portion 26, a third portion 30 that extends from the second portion 28, and a fourth portion 32 that extends from the third portion 30. The first, second, and third portions 26, 28, and 30 can be cylindrical and concentric, and the fourth portion 32 can be a rectangular cube or another non-cylindrical shape for receipt in a drive tool such as a socket. As shown in FIG. 2, the first portion 26 can have a first diameter D1, the second portion 28 can have a second diameter D2 that is less than the first diameter D1, and the third portion 30 can have a third diameter D3 that is less than the second diameter D2. Thus, the central peg 14 can decrease in diameter from the first portion 26 to the third portion 30 in a stepped manner, which may strengthen a press fit between the central peg 14 and a corresponding hole in a scapula. Alternatively, the diameter of the central peg 14 can be decreased in a tapered manner to strengthen the press fit.

The implant 10 can be formed from any biocompatible material, including, polymer, ceramic, metal or combinations thereof. The implant 10 can be formed using additive manufacturing, which enables forming multiple implants in a single build and to decrease manufacturing time. Once formed, the implant 10 can be further processed (e.g., polished, blasted, machining) as desired. For example, the articular surface 20 can be polished for articulation with a humeral head made from polyethylene or another suitable material. Alternatively, polyethylene can be molded over or pressed onto the body 12 to form the articular surface 20 for articulation with a metal humeral head.

The implant 10 is configured to be fixed to a scapula without using fixation hardware such as bone screws. For example, the central peg 14 and the peripheral pegs 16 can be press fit into holes formed into a glenoid cavity to fix the implant to the scapula. In this regard, the central peg 14 and the peripheral pegs 16 may be referred to as fixation members. In addition, annular grooves 34 can be formed into the peripheral pegs 16 for receiving bone cement to fix the peripheral pegs 16 within corresponding holes in the scapula.

The implant 10 is also configured to be fixed to a scapula with minimal bone removal. In this regard, the scapula-engaging surface 22 of the implant 10 can be patient-specific and can be configured to closely conform to as a mirror-image or negative of a surface of a scapula of a specific patient, such as an articular surface of a glenoid cavity, such that the implant 10 nestingly engages the scapula in only one orientation. A 3D model of the scapula can be obtained using an x-ray, MRI, CT, ultrasound or other medical scan, and the scapula-engaging surface 22 can be designed (e.g., shaped, sized, contoured) based on the 3D model. The scapula-engaging surface 22 can be a flat or planar surface as shown when, for example, a corresponding worn or fractured surface of a glenoid cavity of a specific patient is smooth or faceted. Alternatively, the scapula-engaging surface 22 can be a curved, irregular, or non-planar surface.

In addition, the implant 10 can include a bone-filling buildup or protrusion 36 that extends from the scapula-engaging surface 22. The protrusion 36 can be configured to fill a defect, void, or cavity that extends into the scapula such that the cavity is recessed relative to the surface of the scapula to which the scapula-engaging surface 22 is configured to conform. A 3D model of the scapula can be obtained using an x-ray, MRI, CT, ultrasound or other medical scans, and the protrusion 36 can be designed to mirror the cavity in the scapula based on the 3D model. For example, the size, shape, and location of the protrusion 36 can match the size, shape, and location of the defect in the scapula. To this end, as shown in FIG. 4, the protrusion 36 can have a height H1 and a width W1 that are equal to the height and width of the cavity, respectively. The protrusion 36 can be formed integral with the body 12, or the protrusion 36 can be formed separate from the body 12 and attached to the body 12 using, for example, a press-fit and/or an adhesive. Although the implant 10 is shown with only one protrusion, the implant 10 may include additional or fewer protrusions.

The protrusion 36 can extend to the perimeter of the body 12, as shown, or the protrusion 36 can be disposed entirely within and spaced apart from the perimeter of the body 12. As shown in FIG. 2, the protrusion 36 includes a first surface 38, a second surface 40, and a third surface 42. The first surface 38 is configured to closely conform to as a mirror-image or negative of a bottom surface of the cavity. If the protrusion 36 is integrally formed with the body 12, the first surface 38 may form part of the scapula-engaging surface 22. The second surface 40 acts as a transition from the first surface 38 to the peripheral surface 18 of the body 12. The third surface 42 acts as a transition from the first surface 38 to the scapula-engaging surface 22 of the body 12.

The first surface 38 of the protrusion 36 can be a flat or planar surface, as shown, or the first surface 38 can be a curved, irregular, or non-planar surface. The second surface 40 of the protrusion 36 can be aligned with and follow the contour of the peripheral surface 18 of the body 12. In this regard, the second surface 40 of the protrusion 36 and the peripheral surface 18 of the body 12 together may form a portion of the peripheral surface of the implant 10. The third surface 42 may be perpendicular to the scapula-engaging surface 22 of the body 12 to yield a stepped transition from the scapula-engaging surface 22 of the body 12 to the first surface 38 of the protrusion 36.

An articular surface of a glenoid cavity may be worn unevenly such that the general contour of the articular surface is different from its original form. A non-custom or standard glenoid implant may have a convex surface for mating with a reamed articular surface of a glenoid cavity, and the convex surface may not be designed for a specific patient. For example, the general contour of the convex surface may be different from the general contour of a worn articular surface of a glenoid cavity of a specific patient. Thus, when the standard implant is positioned against the worn articular surface of the glenoid cavity, the articular surface of the glenoid implant may be oriented at an anatomically incorrect angle (e.g., a different version and/or inclination angle relative to the original articular surface of the glenoid cavity). In turn, the stress on the implant may be relatively high, which may increase the likelihood of the implant loosening from the bone. In addition, central and/or peripheral pegs of the standard glenoid implant may perforate or puncture through the backside of the scapula.

In addition, the articular surface of the glenoid cavity may have irregularities or defects caused by, for example, wear due to articulation with a humeral head. However, a non-custom or standard glenoid implant may have a convex surface for mating with a reamed articular surface of a glenoid cavity, and the convex surface may not be configured to mate with or seat against an irregular articular surface of the glenoid cavity. Thus, the articular surface of the glenoid cavity may be reamed to eliminate irregularities of the planar surface and/or to orient an articular surface of the standard implant at an anatomically correct angle (e.g., an angle that matches the orientation of the original articular surface of the glenoid cavity), leaving less bone to which the glenoid implant may be attached.

In contrast, the scapula-engaging surface 22 of the implant 10 can be configured to closely conform to as a mirror-image or negative of an articular surface of a glenoid while orienting the articular surface 20 of the implant 10 at an anatomically correct angle. In addition, the protrusion 36 extending from the scapula-engaging surface 22 may fill a defect or cavity that is recessed relative to the articular surface of the glenoid. Thus, the implant 10 may require little to no reaming to seat the scapula-engaging surface 22 of the implant 10 against the articular surface of the glenoid cavity while orienting the articular surface 20 of the implant 10 at an anatomically correct angle. Thus, using the implant 10 may retain more bone for implant fixation relative to using a standard implant.

In some instances, if a glenoid has a defect or cavity that is recessed relative to an articular surface of the glenoid, bone graft may be used to fill the cavity before attaching a standard glenoid implant to the scapula. However, if the standard glenoid implant is press fit or cemented into the glenoid, the amount of compression on the bone graft may be relatively low. In turn, the bone graft may change shape over time such that the bone graft does not completely fill the cavity, which may increase the likelihood of the implant loosening from the bone. In contrast, the protrusion 36 may be made from a harder material than bone graft that does not require compression to maintain its shape, and therefore the implant 10 may be less likely to loosen from the bone.

The scapula-engaging surface 22 may be patient-specific in varying degrees. In one example, the scapula-engaging surface 22 may follow the general contour of an articular surface of a glenoid cavity, but may not closely conform to as a mirror-image or negative of irregularities or defects in the articular surface. Thus, a minimal amount of bone (e.g., from 1 millimeter (mm) to 2 mm) may be removed from the articular surface to eliminate the irregularities or defects such that scapula-engaging surface 22 seats against the articular surface. In this regard, the entire scapula-engaging surface 22 may be configured to mate with an altered surface of a glenoid cavity.

In a second example, a first portion of the scapula-engaging surface 22 may be configured to mate with an unaltered surface of a glenoid caivty, and a second portion of the scapula engaging surface 22 can be configured to mate with an altered surface of the glenoid cavity. The first portion of the scapula-engaging surface 22 may comprise one or more of the surfaces 38, 40, and 42 of the protrusion 36, and the second portion of the scapula-engaging surface 22 may comprise the remainder of the scapula-engaging surface 22. The second portion of the scapula-engaging surface 22 may not be patient-specific or may follow the general contour of a glenoid articular surface of a specific patient. A glenoid articular surface may be reamed to a depth (e.g., from 1 mm to 2 mm) that accommodates the second portion of the scapula-engaging surface 22. Since the protrusion 36 may be configured to fill a void or defect having a depth (e.g., 10 mm) that is greater than the reamed depth, the surface of the defect may be left unaltered. Thus, instead of reaming the glenoid articular surface by an amount that is sufficient to eliminate the defect, only a minimal amount of bone may be removed from the glenoid articular surface.

In a third example, the entire scapula-engaing surface 22 may be configured to mate with an unaltered surface of a glenoid cavity. As with the last example, the scapula-engaging surface 22 may include a first portion comprising one or more of the surfaces 38, 40, 42 of the protrusion 36 and a second portion comprising the remainder of the scapula-engaging surface 22. However, in contrast to the last example, both the first and second portions of the scapula-engaging surface 22 may be configured to mate with an unaltered surface of a glenoid cavity. Thus, the entire glenoid articular surface may be left unaltered. The first portion of the scapula-engaging surface 22 can be configured to conform to as a mirror-image or negative of irregularities or defects in the articular surface having a relatively small depth (e.g., from 1 mm to 2 mm). The second portion of the scapula-engaging surface 22 can be configured to fill a defect in the articular surface having a relatively large depth (e.g., 10 mm).

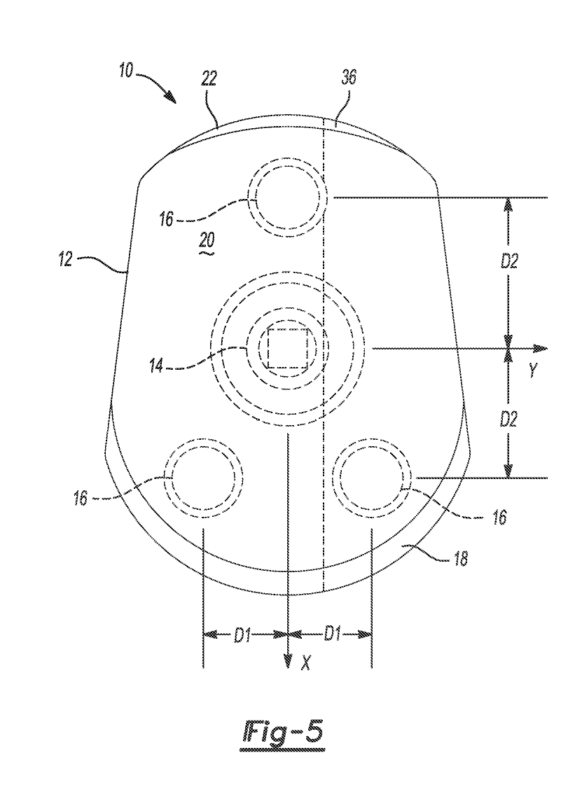

Various other aspects of the implant 10 may be patient-specific. As shown in FIG. 3, the central peg 14 may have a patient-specific length L1 and be oriented at a patient-specific angle A1 relative to the scapula-engaging surface 22 of the body 12. In addition, the diameters D1, D2, and D3 of the central peg 14 and the location of the central peg 14 may be patient-specific. Further, the peripheral pegs 16 may have a patient-specific length L2 and a patient-specific diameter D4, and be oriented at a patient-specific angle A2 relative to the scapula-engaging surface 22 of the body 12. In addition, the location of the peripheral pegs 16 may be patient-specific. For example, as shown in FIG. 5, the peripheral pegs 16 may be located at a distance D1 from a longitudinal axis X of the body 12 and a distance D2 from a lateral axis Y of the body 12. Moreover, the overall size and shape of the implant 10 may be patient-specific and the final (i.e., implanted) version or inclination of the implant 10 may be patient-specific.

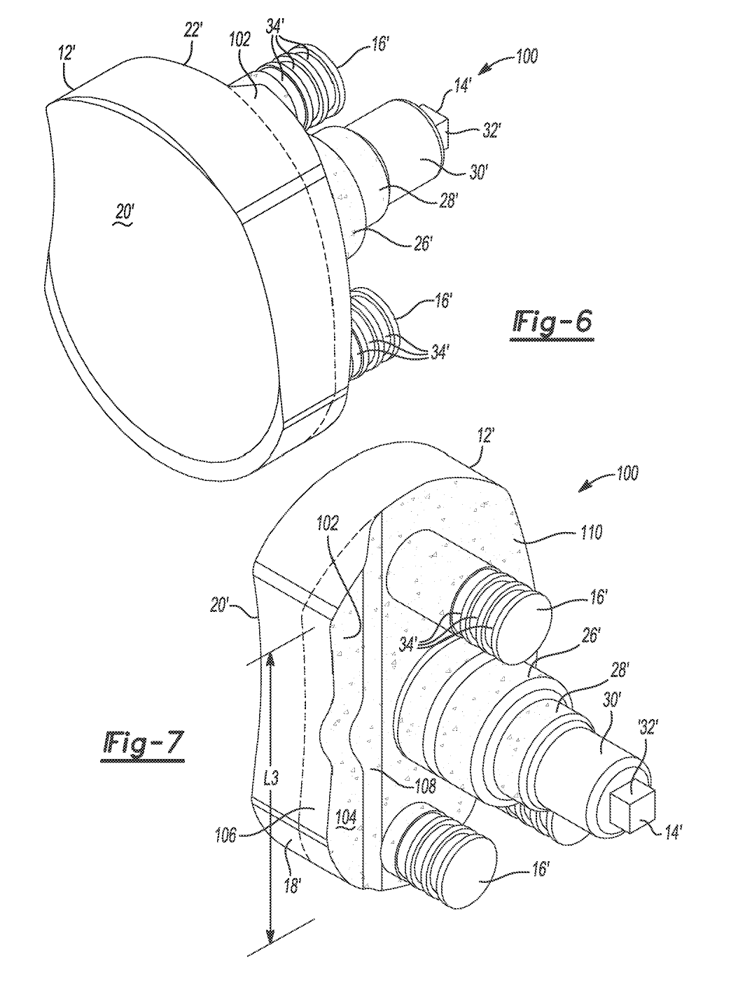

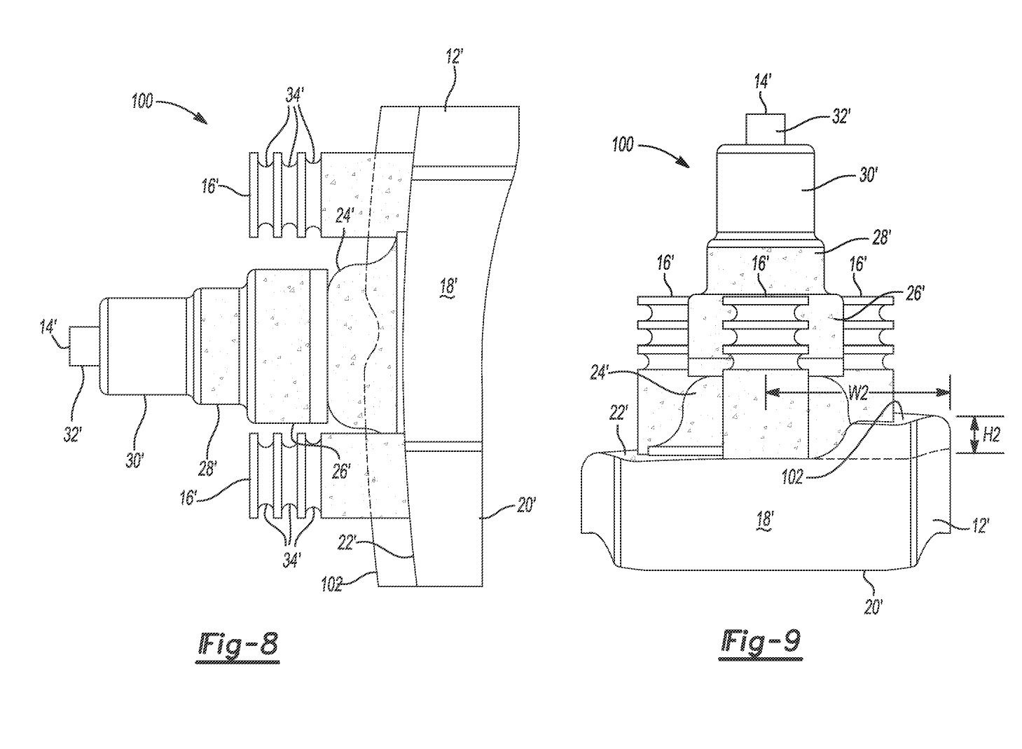

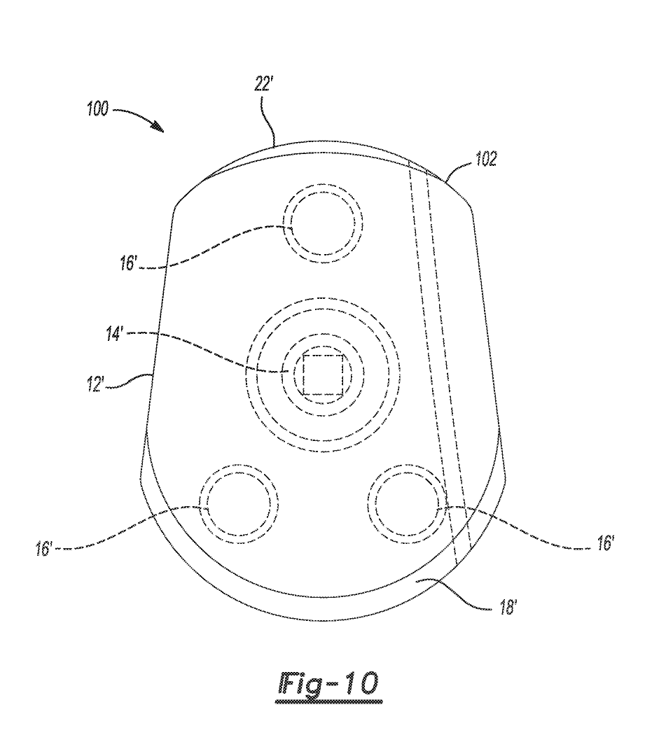

Referring now to FIGS. 6 through 10, a glenoid implant 100 is similar to the implant 10 such that only differences between the implants 10, 100 will now be described. The implant 100 includes a bone-filling or void-filling protrusion 102 extending from the scapula-engaging surface 22. Like the bone-filling protrusion 36, the protrusion 102 can be configured to fill a cavity in a scapula and to closely conform to as a mirror-image or negative of surface(s) of the cavity based on a 3D model of the scapula without modifying the surface(s). However, the protrusion 102 has a patient-specific width W2 (FIG. 9) that is less than the width W1 of the protrusion 36, and the protrusion 102 has a patient-specific height H2 that varies along a length L3 (FIG. 7) of the protrusion 102.

As shown in FIG. 7, the protrusion 102 has a first surface 104, a second surface 106, and a third surface 108. The first surface 104 is configured to closely conform to as a mirror-image or negative of a bottom surface of the cavity. For example, the first surface 104 can be a curved, irregular, or non-planar surface, as shown, resulting in the variable height H2 of the protrusion 36. The second surface 106 acts as a transition from the first surface 104 to the peripheral surface 18′ of the body 12′. The second surface 106 can be aligned with and follow the contour of the peripheral surface 18′ of the body 12′. The third surface 108 acts as a transition from the first surface 104 to the scapula-engaging surface 22′ of the body 12′. The third surface 108 may be angled relative to the scapula-engaging surface 22′ of the body 12′ to yield a ramped transition from the scapula-engaging surface 22′ to the first surface 104.

A porous coating 110 can be applied to one or more areas or surfaces of the implant 100 to maximize porous in-growth, while solid material may be used in areas under high loads to provide support. The porous coating 110 can be applied to the central peg 14′, the peripheral pegs 16′, the scapula-engaging surface 22′ of the body 12′, the first surface 104 of the protrusion 102, and the third surface 108 of the protrusion 102, as shown. The location of the porous coating 110 may be patient-specific. For example, the porous coating 110 may be applied to areas of the implant 100 that contact the glenoid, which may be determined based on 3D models of the implant 100 and the glenoid.

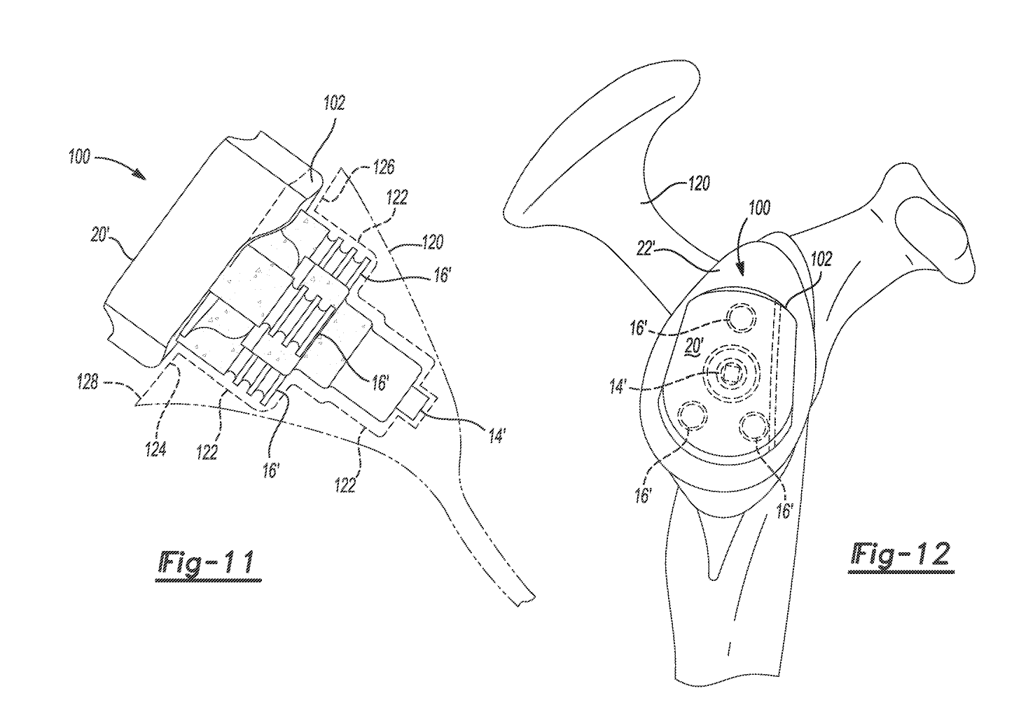

Referring now to FIGS. 11 and 12, an example method of repairing a glenoid cavity will now be described. First, a surgeon takes an x-ray, MRI, CT, ultrasound or other medical scan of a scapula 120 of a specific patient. The medical scan is loaded into a software program configured to generate a 3D model of the scapula 120 based on the medical scan. Further discussion of generating a 3D model of a patient's anatomy using a medical scan can be found in U.S. Pat. Pub. No. 2013/0110470, which is incorporated herein by reference.

The surgeon then determines whether a non-custom implant is compatible with the scapula 120 based on the 3D model of the scapula 120. For example, the surgeon may determine whether an amount of bone removal required to seat the non-custom implant against the scapula 120 is less than a predetermined amount. In addition, the surgeon may determine whether the non-custom implant yields a desired version when fixed to the scapula 120.

If the non-custom implant is compatible with the scapula 120, the surgeon fixes the non-custom implant to the scapula 120. Otherwise, the implant 100 may be designed to fit the scapula 120 based on the 3D model of the scapula 120. The implant 100 may be designed by the surgeon, a third party (e.g., an implant manufacturer), automatically by software, or a combination thereof. The implant 100 may be designed by modifying a 3D model of a non-custom implant or creating a 3D model of the implant 100 to include one or more of the patient-specific features described above, such as a bone-filling protrusion configured to match as a mirror-image of an unaltered bone surface. The patient-specific instruments and implants may be packaged as a single-use kit and sent to the surgeon to simplify the shoulder joint replacement procedure.

The implant 100 may then be formed and fixed to the scapula 120. Before fixing the implant 100 to the scapula 120, the surgeon may drill holes 122 in the scapula 120 for receiving the central peg 14′ and the peripheral pegs 16′. The holes 122 can be sized to yield a press fit between the implant 100 and the scapula 120 or oversized relative to the pegs 14′, 16′ to allow for cement fixation. The surgeon may also ream a portion of the scapula 120 to conform to the body 12′, although this may not be required as the body 12′ can be designed to conform to the scapula 120 without reaming the scapula 120.

The instruments (e.g., drills, guides, reamers) used by the surgeon to prepare the scapula 120 may be patient-specific. For example, the patient-specific instruments can have a three-dimensional engagement surface that is complementary and made to conformingly contact the anatomical surface. Thus, the patient-specific instruments can be configured to fit in only one position to the anatomical surface. Further discussion of patient-specific instruments can be found in U.S. Pat. Pub. No. 2013/0110116, U.S. Pat. Pub. No. 2013/0110470, and U.S. patent application Ser. No. 13/653,893, which are incorporated herein by reference

When the implant 100 is fixed to the scapula 120, the scapula-engaging surface 22′ of the body 12′ conforms to and mirrors a corresponding surface 124 of the scapula 120. In addition, the protrusion 102 fills a defect 126 in the surface 124 such as a glenoid rim fracture. Further, the articular surface 20′ of the implant 100 can form a smooth, continuous surface with portions of a glenoid cavity 128 surrounding the articular surface 20′. The articular surface 20′ and the surrounding portions of the glenoid cavity 128 can accurately reproduce the natural movement of the shoulder joint including glenoid version.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claims is:

1. A method of manufacturing a glenoid implant, comprising:
obtaining a three-dimensional (3D) model of a scapula of a specific patient;
designing the glenoid implant to have an articular surface and a scapula-engaging surface opposite from the articular surface that mirrors and conforms to a surface of the scapula based on the 3D model of the scapula such that the glenoid implant nestingly engages the scapula in only one orientation; and
forming the glenoid implant.

2. The method of claim 1, wherein designing the glenoid implant includes designing a patient-specific, bone-filling protrusion to extend from the scapula-engaging surface of the glenoid implant and be configured to fill a defect in the surface of the scapula based on the 3D model of the scapula.

3. The method of claim 1, wherein designing the glenoid implant includes designing a fixation member to extend from the scapula-engaging surface and have a length, diameter, orientation, and location that are patient-specific.

4. The method of claim 1, wherein designing the glenoid implant includes designing a size, shape, placement, and inclination angle of the glenoid implant to be patient-specific.

5. The method of claim 1, wherein forming the glenoid implant includes forming the glenoid implant using additive manufacturing.

6. A method of repairing a glenoid, comprising:
obtaining a three-dimensional (3D) model of a scapula of a specific patient;
determining whether a non-custom implant is compatible with the glenoid based on the 3D model of the glenoid; and
based on the determination, fixing one of the non-custom implant and a patient-specific implant to the glenoid, wherein the patient-specific implant has a patient-specific bone-engaging surface.

7. The method of claim 6, wherein determining whether the non-custom implant is compatible with the glenoid includes determining whether an amount of bone removal required to seat the non-custom implant against the glenoid is less than a predetermined amount.

8. The method of claim 6, wherein determining whether the non-custom implant is compatible with the glenoid includes determining whether the non-custom implant yields a desired version when seated against the glenoid.
